(19) United States
(12) Patent Application Publication
(10) Pub. No.: US 2025/0228538 A1
(43) Pub. Date: Jul. 17, 2025

(54) DEVICES, SYSTEMS, AND METHODS DEVICE FOR DELIVERY OF A MULTI-COMPONENT SEALANT

(71) Applicant: Bard Peripheral Vascular, Inc., Franklin Lakes, NJ (US)

(72) Inventors: Jordan ADDISON, Tempe, AZ (US); Heather A. STORM, Vernon Hills, IL (US); Luke WINTERSTEIN, Tempe, AZ (US)

(73) Assignee: Bard Peripheral Vascular, Inc., Franklin Lakes, NJ (US)

(21) Appl. No.: 18/854,218

(22) PCT Filed: Apr. 6, 2022
(86) PCT No.: PCT/US2022/023630

§ 371 Date: Oct. 4, 2024

(51) Int. Cl. A61B 17/00; A61B 17/34

(52) U.S. Cl. CPC A61B 17/00491; A61B 2017/00495; A61B 2017/3407; A61B 2017/3409

(57) ABSTRACT

A medical sealant applicator device for delivery of a multi-component sealant including a first body having a base, a first syringe plunger, a second syringe plunger, and a longitudinal channel, laterally interposed between the first syringe plunger and the second syringe plunger. Each of the first syringe plunger and the second syringe plunger is oriented to extend in a first direction from the base. The first syringe plunger has a first free end having a first proximal plunger piston. The second syringe plunger having a second free end having a second proximal plunger piston. A second body of the medical sealant application device has a first syringe chamber, a second syringe chamber, and a longitudinal fluid chamber laterally interposed between the first syringe chamber and the second syringe chamber. The longitudinal fluid chamber has a proximal end and a distal end.

### TECHNICAL FIELD

The present disclosure generally relates to delivery of sealants for a medical procedure and more specifically, to devices, systems, and methods for delivering a multi- component sealant in a medical procedure.

### BACKGROUND

In lung biopsy procedures, a potential concern for medical professionals is preventing or reducing the likelihood of air and/or fluid entry into the pleural space within the lung. This condition is known as pneumothorax and may occur as a result of the puncture created during or after the lung biopsy procedure. Pneumothorax is a common occurrence and results in an increased hospital stay. Additionally, subjects may require a chest tube in response to pneumothorax occurring. Medical personnel may find it difficult to seal the puncture after the procedure due to varying complications as a result of the pneumothorax.

Apparatus and methods have been used to attempt to seal the puncture. However, these apparatus and methods are limited in that medical personal may only be able to use a single-component sealant and/or may not have an ability to adjust a rate of sealant delivery.

### SUMMARY

In accordance with an embodiment of the disclosure, there is provided a medical sealant applicator device for delivery of a multi-component sealant including a first body having a base, a first syringe plunger, a second syringe plunger, and a longitudinal channel, laterally interposed between the first syringe plunger and the second syringe plunger. Each of the first syringe plunger and the second syringe plunger is oriented to extend in a first direction from the base. The first syringe plunger has a first free end having a first proximal plunger piston. The second syringe plunger has a second free end having a second proximal plunger piston. A second body of the medical sealant application device has a first syringe chamber, a second syringe chamber, and a longitudinal fluid chamber laterally interposed between the first syringe chamber and the second syringe chamber. The longitudinal fluid chamber has a proximal end and a distal end. The proximal end of the longitudinal fluid chamber is connected in fluid communication with each of the first syringe chamber and the second syringe chamber. The distal end of the longitudinal fluid chamber has a distal needle connector port. The first syringe chamber of the second body has a first distal opening to slidably receive the first proximal plunger piston of the first syringe plunger of the first body. The second syringe chamber of the second body has a second distal opening to slidably receive the second proximal plunger piston of the second syringe plunger of the first body, and the distal needle connector port of the longitudinal fluid chamber of the second body is able to translate in the longitudinal channel of the first body in response to the second body longitudinally translating relative to the first body.

In yet another embodiment, there is provided a medical sealant applicator device for delivery of a multi-component sealant, includes a first body, a second body, and a guide assembly. The first body has a base, a first syringe plunger, a second syringe plunger, and a longitudinal channel laterally interposed between the first syringe plunger and the second syringe plunger. Each of the first syringe plunger and the second syringe plunger is oriented to extend in a first direction from the base. The first syringe plunger has a first free end having a first proximal plunger piston. The second syringe plunger has a second free end having a second proximal plunger piston. The second body has a first syringe chamber, a second syringe chamber, and a longitudinal fluid chamber laterally interposed between the first syringe chamber and the second syringe chamber. The longitudinal fluid chamber has a proximal end and a distal end. The proximal end of the longitudinal fluid chamber is connected in fluid communication with each of the first syringe chamber and the second syringe chamber. The distal end of the longitudinal fluid chamber has a distal needle connector port. The first syringe chamber of the second body has a first distal opening to slidably receive the first proximal plunger piston of the first syringe plunger of the first body. The second syringe chamber of the second body has a second distal opening to slidably receive the second proximal plunger piston of the second syringe plunger of the first body. The distal needle connector port of the longitudinal fluid chamber of the second body is able to translate in the longitudinal channel of the first body in response to the second body longitudinally translating relative to the first body. The medical sealant applicator device further includes: a guide assembly. The guide assembly sets a trajectory towards a targeted location and maintains the trajectory while advancing the needle assembly and injecting a component.

In yet another embodiment, there is provided a method includes setting a trajectory for a needle assembly through a pleural space towards a target at a known distance and maintaining the trajectory while advancing the needle assemble. The method further includes advancing the needle assembly at a first rate through the pleural space to the known distance. The method further includes applying a force to a proximal end of the needle assemble in a distal direction, wherein the force simultaneously injects a substance at a second rate.

In yet another embodiment, a component delivery system, includes a needle assembly, an applicator, a first body, and a second body. The needle assembly has an elongate needle comprising a proximal end and a distal end spaced a distance apart from one another and defining at least one fluid path therebetween. The distal end comprising one or more fluid outlets coupled to the at least one fluid path. The applicator being fluidly coupled to the proximal end of the needle assembly. The applicator includes a first body comprising a first biasing assembly that is coupled to the needle assembly. The first body biasing the needle assembly in a proximal direction with respect to the applicator. The second body includes a syringe chamber having a needle connector port on a proximal end thereof that is fluidly coupled to the needle assembly, and a syringe plunger disposed within the syringe chamber at a distal end thereof and movable within the syringe chamber. The second biasing assembly that is coupled between the first body and the second body, the second biasing assembly biasing the syringe plunger at the distal end of the syringe chamber.

These and additional features provided by the embodiments described herein will be more fully understood in view of the following detailed description, in conjunction with the drawings.

### BRIEF DESCRIPTION OF THE DRAWINGS

FIG. 1 is a perspective view of a component delivery system including a protective spacer, according to one or more embodiments shown and described herein;

FIG. 2 is a front view of the component delivery system of FIG. 1, according to one or more embodiments shown and described herein;

FIG. 3A is a detailed view of the component delivery system taken along section 3A in FIG. 2, according to one or more embodiments shown and described herein;

FIG. 3B is a detailed view of the component delivery system with select components omitted, according to one or more embodiments shown and described herein;

FIG. 4 is a perspective view of a guide assembly, according to one or more embodiments shown and described herein;

FIG. 5 is a front view of a threaded shaft of a guide assembly, according to one or more embodiments shown and described herein;

FIG. 6 is a top view of the threaded shaft of FIG. 5, according to one or more embodiments shown and described herein;

FIG. 7 is a perspective view of the guide assembly of FIG. 4 with select components omitted, according to one or more embodiments shown and described herein;

FIG. 8 is another perspective view of the guide assembly of FIG. 4 with select components omitted, according to one or more embodiments shown and described herein;

FIG. 9 is another perspective view of the guide assembly of FIG. 4 with select components omitted, according to one or more embodiments shown and described herein;

FIG. 10 is a detailed view of the guide assembly taken along section 10 in FIG. 4, according to one or more embodiments shown and described herein;

FIG. 11 is a perspective view of a component delivery system and a guide assembly at a first position, according to one or more embodiments shown and described herein; and

FIG. 12 is a perspective view of a component delivery system and a guide assembly at a second position, according to one or more embodiments shown and described herein.

### DETAILED DESCRIPTION

Reference will now be made in detail to various embodiments of component delivery systems for administering sealant to a subject, examples of which are illustrated in the accompanying drawings. Whenever possible, the same reference numerals will be used throughout the drawings to refer to the same or like parts. Directional terms as used herein—for example up, down, right, left, front, back, top, bottom, distal, and proximal—are made only with reference to the figures as drawn and are not intended to imply absolute orientation.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

Unless otherwise defined, all technical and scientific terms used herein have the same meaning as commonly understood by one of ordinary skill in the art to which the disclosure belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

As used herein, the terms “horizontal,” “vertical,” “distal” and “proximal” are relative terms only, are indicative of a general relative orientation only, and do not necessarily indicate perpendicularity. These terms also may be used for convenience to refer to orientations used in the figures, which orientations are used as a matter of convention only and are not intended as characteristic of the devices shown. The present disclosure and the embodiments thereof to be described herein may be used in any desired orientation. Moreover, horizontal and vertical walls need generally only be intersecting walls, and need not be perpendicular. As used herein, the singular forms “a,” “an” and “the” include plural referents unless the context clearly dictates otherwise. Thus, for example, reference to “a” component includes aspects having two or more such components, unless the context clearly indicates otherwise.

During lung biopsies procedures, a potential concern for medical professionals is the likelihood of air and/or fluid entry into the pleural space within the lung. To mitigate this, the present disclosure is directed to a component delivery system that seals the path made by a needle assembly, while the needle assembly is injected into the patient. When the component delivery system uses a multi-component sealant, the component delivery system controls a flow rate of each component of the multi-component sealant so that the multi-component sealant has the correct ratio. The component delivery system may be placed into a guide assembly. The guide assembly is placed onto the subject. The guide assembly allows for the medical professional to control a height for the component delivery system and the angle of the component delivery system.

The component delivery system has the technical effect that in response to a medical professional applying the component delivery system, a first body of the component delivery system is pushed towards a second body of the component delivery system. The second body includes each component of the multi-component sealant. After the second body is pushed into the first body, each component is dispensed from the first body to a mixing chamber. In the mixing chamber, the multi-component sealant begins to mix and is then dispensed from a needle assembly of the component delivery system.

The guide assembly has the technical effect of controlling a depth and angle for a needle assembly prior to being injected into a subject. The depth control of the guide assembly moves the needle assembly along a single direction. The angle control changes an entry angle of the needle assembly. Adjusting the angle of the needle assembly does not impact the depth of the guide assembly.

### I. Component Delivery System

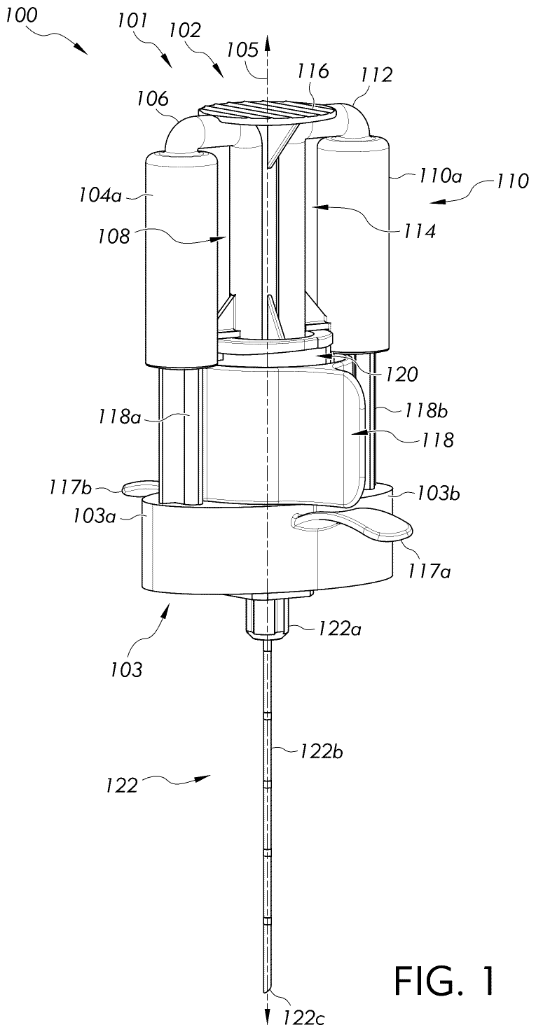

Referring now to FIGS. 1-3B, a component delivery system 100 is shown, according to some embodiments. The component delivery system 100 may be used during a medical procedure and/or during a medical study. The component delivery system 100 includes an applicator 101, a mixing chamber 120, and a needle assembly 122 fluidly coupled to the applicator 101.

The applicator 101 stores each component of the multi-component sealant. In response to receive a biasing force, the applicator 101 provides each component of the multi- component sealant to the mixing chamber 120. The applicator 101 includes a first body 103 and a second body 102. The first body 103 is positioned at a first axial end of the applicator 101 and is centered on a center axis 105 of the component delivery system 100. The second body 102 is positioned at a second axial end of the applicator 101 and is also centered on the center axis 105. As discussed herein, the first body 103 co-operates with the second body 102 to provide each component of the multi-component to the mixing chamber 120 in response to receiving the biasing force. Specifically, the biasing force results in each component of the multi-component sealant to flow from the applicator 101 to the needle assembly 122 while maintaining a controlled flow rate.

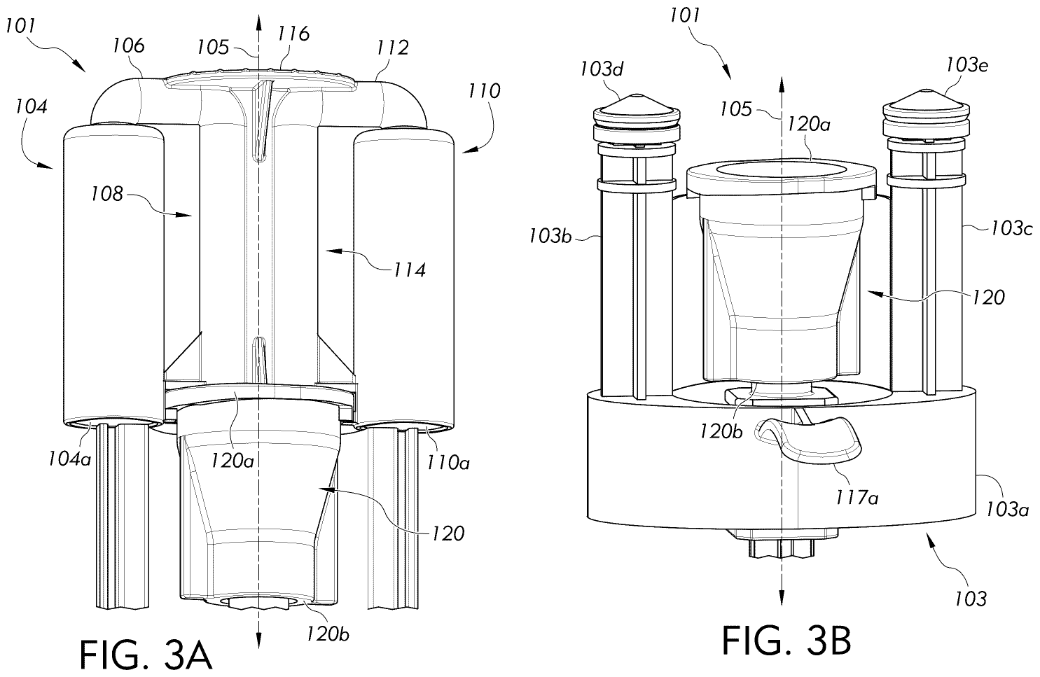

Now referring to FIG. 3B, the first body 103 includes a base 103a, a first syringe plunger 103b, a second syringe plunger 103c, a first finger-hold projection 117a, and a second finger-hold projection 117b. As depicted, the base 103a defines an oval shape but other geometries are contemplated and possible (e.g., rectangular, triangular, hexagonal). The base 103a defines a distal aperture extending through a depth of the base 103a and is centered along the center axis 105, thereby providing space for the mixing chamber 120 and needle assembly 122 to extend through. The base 103a may be constructed of plastic, metal, or any other suitable material.

The first syringe plunger 103b and the second syringe plunger 103c extend from a first surface of the base 103a and are further positioned on opposing sides of the first surface of the base 103a. Each of the first syringe plunger 103b and the second syringe plunger 103c may be coupled to the base 103a (e.g., adhesion, fastening, plastic welding) or integrated with the base 103a (e.g., machining, molding, forming, plastic injection). Each of the first syringe plunger 103b and the second syringe plunger 103c may be parallel to the center axis 105.

The first syringe plunger 103b includes a first plunger top 103d and the second syringe plunger 103c includes a second plunger top 103e. Each plunger top 103d, 103e may be constructed of rubber, plastic or any suitable material. The first syringe plunger 103b is in contact with a first component of the multi-component sealant via the first plunger top 103d and the second syringe plunger 103c is in contact with a second component of the multi-component sealant via the second plunger top 103e. In response to a force being applied upon the first body 103, each of the first syringe plunger 103b and the second syringe plunger 103c applies a force upon the respective component of the multi-component sealant. As depicted, the first syringe plunger 103b and the second syringe plunger 103c are piston-type rods defining a cone-shaped top. However, any suitable syringe plunger may be utilized.

The first finger-hold projection 117a, and the second finger-hold projection 117b laterally extend from the first body 103 in a direction perpendicular to the center axis 105. The first finger-hold projection 117a and the second finger-hold projection 117b each define a curved profile. The curved profile is shaped to provide an ergonomic feel for a medical professional, such that a medical professional's first finger may apply a force or hold onto the first finger-hold projection 117a, and a medical professional's second finger may apply a force or hold onto the second finger-hold projection 117b. Although depicted as a curved profile, other geometric shapes are contemplated and possible. For example, the finger-hold projections 117a, 117b may be enclosed circles.

The force may be applied in a proximal direction along the center axis 105 and towards the second body 102. In embodiments, the first finger-hold projection 117a and the second finger-hold projection 117b may be used to steady (e.g., stabilize) the base 103a when applying the applicator 101. In embodiments, more than two finger-hold projections are used.

The second body 102 is positioned on an axial end of the applicator 101 opposing the first body 103. The second body 102 is centered on the center axis 105 and may be constructed of any suitable material such as plastic, metal, glass and the like.

The second body 102 includes a first syringe barrel 104, a first u-shaped connector 106, a first longitudinal barrel 108, a second syringe barrel 110, a second u-shaped connector 112, a second longitudinal barrel 114, and an engaging feature 116.

The engaging feature 116 is positioned on the second axial end of the applicator 101 and is centered on the center axis 105. As depicted, the engaging feature 116 defines a flat profile, however, other geometric profiles are contemplated and possible (e.g., concave profile, convex profile). The engaging feature 116 is shaped to provide an ergonomic profile such that a finger of the medical professional may easily apply a force onto the engaging feature 116 to apply a force on the second body 102 along the center axis 105 towards the first body 103. As the engaging feature 116 is centered on the center axis 105, thereby being concentric to the second body 102, a force placed onto the engaging feature 116 is distributed equally onto the second body 102. The engaging feature 116 may define a plurality of ridges to help a medical professional engage with the engaging feature 116.

To apply the multi-component delivery system 100 a force is placed onto the applicator 101. In embodiments, a force is placed on the engaging feature 116, thereby causing the second body 102 to translate towards the first body 103. In embodiments, a force is placed upon the first finger-hold projection 117a, and the second finger-hold projection 117b, thereby causing the first body 103 to translate towards the second body 102.

The first syringe barrel 104 contains a first component of the multi-component sealant. The first syringe barrel 104 may be translucent, enabling a medical professional to see the first component or opaque to protect the first component from light. The first syringe barrel 104 extends along an axis parallel to the center axis 105.

The first syringe plunger 103b extends into a first syringe aperture 104a of the first syringe barrel 104. The first syringe plunger 103b and the first syringe aperture 104a are co-operatively sized such that the first plunger top 103d of the first syringe plunger 103b creates an air-tight seal in the first syringe barrel 104. This air-tight seal prevents the first component located within the first syringe barrel 104 from leaking out of the first syringe barrel 104 and from exposing the first component to the atmosphere. An inner diameter of the first syringe barrel 104 may be adjusted to control a rate of component exiting the first syringe barrel 104 during application. For example, if the ratio for creating the multi-component seal requires additional amounts of the first component, the inner diameter of the first syringe barrel 104 may be increased. Additionally, if the ratio for creating the multi-component seal requires less amounts of the first component, the inner diameter of the first syringe barrel 104 may be decreased.

In response to a force being applied onto the applicator 101, a biasing force is placed upon the first component in the first syringe barrel 104 via the first syringe plunger 103b. In other words, in response to the engaging feature 116 being pushed towards the first body 103 and/or the finger-hold projections 117a, 117b being pushed towards the second body 102, the first syringe plunger 103b translates within the first syringe barrel 104 and towards the second body 102. The first syringe plunger 103b may then push the first component within the first syringe barrel 104 out of the first syringe barrel 104.

The first syringe barrel 104 is coupled to and fluidly coupled to the first u-shaped connector 106. The first u-shaped connector 106 laterally extends (e.g., perpendicular to the center axis 105) from the first syringe barrel 104 towards the center axis 105. In embodiments, the first u-shaped connector 106 is integrated to the first syringe barrel 104. In response to the first component exiting the first syringe barrel 104 via the first syringe plunger 103b, the first component is fluidly provided to the first u-shaped connector 106. By defining a u-shaped fluid path, the first component is redirected from traveling towards the second body 102 (e.g., within the first syringe barrel 104) to now travel towards the first body 103 (e.g., after exiting the first u-shaped connector 106). The first u-shaped connector 106 prevents bottlenecks from occurring within the fluid path due to its curved shaped. In this way, the fluid may exit the applicator 101 with a constant flow rate. As the first component continues to travel, it may exit the first u-shaped connector 106.

The first u-shaped connector 106 may be coupled to and fluidly coupled to the first longitudinal barrel 108. In embodiments, the first u-shaped connector 106 is integrated into the first longitudinal barrel 108. The first longitudinal barrel 108 may extend along an axis parallel to the first syringe barrel 104 and the center axis 105. By defining a longitudinal profile parallel to the center axis 105, the first component is directed towards the first body 103. The first longitudinal barrel 108 may be constructed of plastic, glass, or any other suitable material.

The second syringe barrel 110 contains a second component of the multi-component sealant. The second syringe barrel 110 may be translucent, enabling a medical professional to see the second component or opaque to protect the second component from light. The second syringe barrel 110 extends along an axis parallel to the center axis 105.

The second syringe plunger 103c extends into a second syringe aperture 110a of the second syringe barrel 110. The second syringe plunger 103c and the second syringe aperture 110a are co-operatively sized such that the second plunger top 103e of the second syringe plunger 103c creates an air-tight seal in the second syringe barrel 110. This air-tight seal prevents the second component located within the second syringe barrel 110 from leaking out of the second syringe barrel 110 and from exposing the second component to the atmosphere. An inner diameter of the second syringe barrel 110 may be adjusted to control a rate of component exiting the second syringe barrel 110 during application. For example, if the ratio for creating the multi-component seal requires additional amounts of the second component, the inner diameter of the second syringe barrel 110 may be increased. Additionally, if the ratio for creating the multi-component seal requires less amounts of the second component, the inner diameter of the second syringe barrel 110 may be decreased.

In response to a force being applied onto the applicator 101, a biasing force is placed upon the second component in the second syringe barrel 110 via the second syringe plunger 103c. In other words, in response to the engaging feature 116 being pushed towards the first body 103 and/or the finger-hold projections 117a, 117b being pushed towards the second body 102, the second syringe plunger 103c translates within the second syringe barrel 110 and towards the second body 102. The second syringe plunger 103c may then push the second component within the second syringe barrel 110 out of the second syringe barrel 110.

The second syringe barrel 110 is coupled to and fluidly coupled to the second u-shaped connector 112. The second u-shaped connector 112 laterally extends (e.g., perpendicular to the center axis 105) from the second syringe barrel 110 towards the center axis 105. In embodiments, the second u-shaped connector 112 is integrated to the second syringe barrel 110. In response to the second component exiting the second syringe barrel 110 via the second syringe plunger 103c, the second component is fluidly provided to the second u-shaped connector 112. By defining a u-shaped fluid path, the second component is redirected from traveling towards the second body 102 (e.g., within the second syringe barrel 110) to now travel towards the first body 103 (e.g., after exiting the second u-shaped connector 112). The second u-shaped connector 112 prevents bottlenecks from occurring within the fluid path due to its curved shaped. In this way, the fluid may exit the applicator 101 with a constant flow rate. As the second component continues to travel, it may exit the second u-shaped connector 112.

The second u-shaped connector 112 may be coupled to and fluidly coupled to the second longitudinal barrel 114. In embodiments, the second u-shaped connector 112 is integrated into the second longitudinal barrel 114. The second longitudinal barrel 114 may extend along an axis parallel to the second syringe barrel 110 and the center axis 105. By defining a longitudinal profile parallel to the center axis 105, the second component is directed towards the first body 103. The second longitudinal barrel 114 may be constructed of plastic, glass, or any other suitable material.

Referring to FIGS. 3A-3B, the mixing chamber 120 is shown positioned between the base 103a and the first longitudinal barrel 108 and the second longitudinal barrel 114. The mixing chamber 120 may be centered along the center axis 105.

As depicted, side walls for the mixing chamber 120 define an inwardly tapering profile such that a mixing inlet 120a defines an area larger than a mixing outlet 120b. In this way, the first component and the second component received by the first longitudinal barrel 108 and the second longitudinal barrel 114, respectively, begin to interface with each other while minimizing opportunities for bottlenecks to occur within the mixing chamber 120. However, other shapes are contemplated and possible. For example, in embodiments, the area of the mixing outlet 120b may be greater than or equal to the area of the mixing inlet 120a. In these embodiments, the side walls of the mixing chamber 120 may define an outwardly tapering profile or may be parallel. The mixing chamber 120 may be constructed of any suitable material such as plastic, ceramic, glass, metal, or any other suitable material.

The first longitudinal barrel 108 and the second longitudinal barrel 114 are fluidly coupled to a mixing inlet 120a of the mixing chamber 120. In response to the first component traveling within the first longitudinal barrel 108 and the second component traveling within the second longitudinal barrel 114, the first component and the second component are provided to the mixing inlet 120a of the mixing chamber 120. Accordingly, in the mixing chamber 120, the first component and the second component may begin to interface and mix together.

In embodiments, the first component and the second component do not mix in the mixing chamber 120. In embodiments, the first component and the second component do begin to mix in the mixing chamber 120 and begin to form the multi-component sealant. In embodiments, the first component and the second component are completely mixed in the mixing chamber 120, thereby forming the multi-component sealant.

In embodiments, the component delivery system 100 does not include the mixing chamber 120. In these embodiments, the first longitudinal barrel 108 and the second longitudinal barrel 114 are integrated together such that the first component and the second component interface within the combined the first longitudinal barrel 108 and the second longitudinal barrel 114. Continuing in these embodiments, the first longitudinal barrel 108 and/or the second longitudinal barrel 114 define a helical profile (not shown) to interface the first component to the second component and vice versa.

Referring now to FIG. 1, the component delivery system 100 is shown with a removable stop 118. To prevent the unintentional application of the applicator 101, the medical professional may assemble the removable stop 118 onto the component delivery system 100. The removable stop 118 is assembled onto the applicator 101 between the first body 103 and the second body 102, thereby preventing the second body 102 from translating towards the first body 103. The removable stop 118 may be placed onto the applicator 101 until the component delivery system 100 is ready to be utilized by the medical professional. In embodiments, the removable stop 118 is integrated to (e.g., molding, casting) the component delivery system 100 and includes removable tabs to allow for easy disassembling of the removable stop 118 from the component delivery system 100. The removable stop 118 may be constructed of plastic, ceramic, metal, or any suitable material.

The removable stop 118 may include a first stopping element 118a and a second stopping element 118b. When the removable stop 118 is assembled onto the applicator 101, the first stopping element 118a is placed upon the first syringe plunger 103b and is positioned between the base 103a and the first syringe barrel 104. Additionally, second stopping element 118b is placed upon the second syringe plunger 103c and is positioned between the base 103a and the second syringe barrel 110. In embodiments, each stopping element 118a, 118b may be removably coupled to each syringe barrel 104, 110 via a snap-fit connection or any suitable like connection. In this way, the removable stop 118 prevents the second body 102 from traveling towards the first body 103 due to a physical interference caused by the first stopping element 118a and the second stopping element 118b. The removable stop 118 may then be removed when it is intended to use the component delivery system 100. In embodiments, the removable stop 118 may include only one of the first stopping element 118a or the second stopping element 118b.

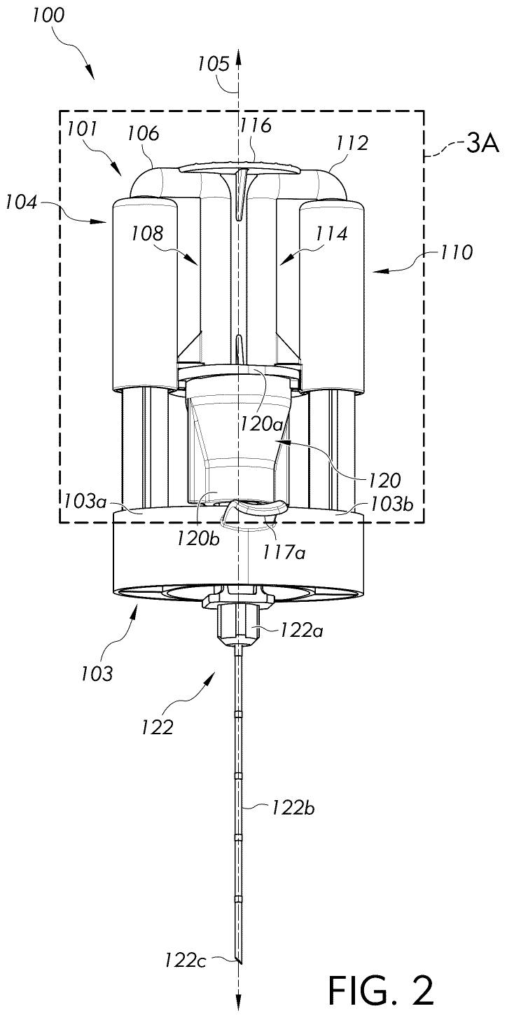

Referring to FIG. 2, the needle assembly 122 is shown including a connector hub 122a and an elongate needle 122b. The needle assembly 122 is centered along the center axis 105 and extends along the center axis 105. The needle assembly 122 dispenses the multi-component sealant from the applicator 101 to a targeted location within the subject. The needle assembly 122 is positioned at an axial end of the component delivery system 100 opposing an opposite axial end of the component delivery system 100 (e.g., where the engaging feature 116 is positioned). The needle assembly 122 may be any available needle assembly used in medical applications. While the needle assembly 122 enters the targeted location of the subject, the needle assembly 122 begins to dispense the multi-component sealant.

The connector hub 122a is coupled to and fluidly coupled to the mixing outlet 120b. The connector hub 122a is centered on the center axis 105. As depicted, the connector hub 122a is embedded into the mixing outlet 120b. However, other fastening methods may be used to couple the connector hub 122a to the mixing outlet 120b, such as fastening threads, adhesion, etc. In embodiments, the connector hub 122a is integrated (e.g., casted, formed, machined, molded) to the mixing chamber 120. As the first component, the second component, and/or the multi-component sealant continue to travel, they exit from the mixing outlet 120b and enter into the connector hub 122a. In embodiments, the first component and the second component may begin mixing or continue to mix within the connector hub 122a.

The elongate needle 122b is coupled to and fluidly coupled to the connector hub 122a. The elongate needle 122b is centered on and extends along the center axis 105. In embodiments, the connector hub 122a may be integrated into the connector hub 122a. In embodiments, the elongate needle 122b receives completely mixed multi-component sealant from the connector hub 122a. In embodiments, the first component and the second component may begin mixing or continue to mix within the elongate needle 122b.

The elongate needle 122b defines a needle outlet 122c. The needle outlet 122c is positioned at an axial end of the elongate needle 122b that is opposite to the connector hub 122a. The multi-component sealant is dispensed to the targeted location within the subject from the elongate needle 122b via the needle outlet 122c.

The component delivery system 100 delivers a multi-component sealant to a subject while a needle assembly of the component delivery system 100 is injected into a pleural space of a subject's lung. In this way, the medical professional seals the path of the needle assembly during the injection of the needle. While injecting the needle assembly 122 into the pleural space of the subject's lung, each component of the multi-component sealant exits a respective syringe barrel in response to the component delivery system 100 experiencing an applied force. Each component of the multi-component sealant is then mixed within the component delivery system 100, thereby forming the multi-component sealant. The multi-component sealant is then dispensed from the needle assembly 122.

In this way, a multi-component sealant may be used due to the configuration of the component delivery system 100 providing a controlled ratio of each component. This is advantageous as multi-component sealants may be superior to single-component sealants for sealing. Additionally, the rate of the component delivery system 100 may be dispensed in a controlled fashion. This is advantageous as it reduces the likelihood of a medical professional applying too much or too little sealant to a subject.

### II. Guide System

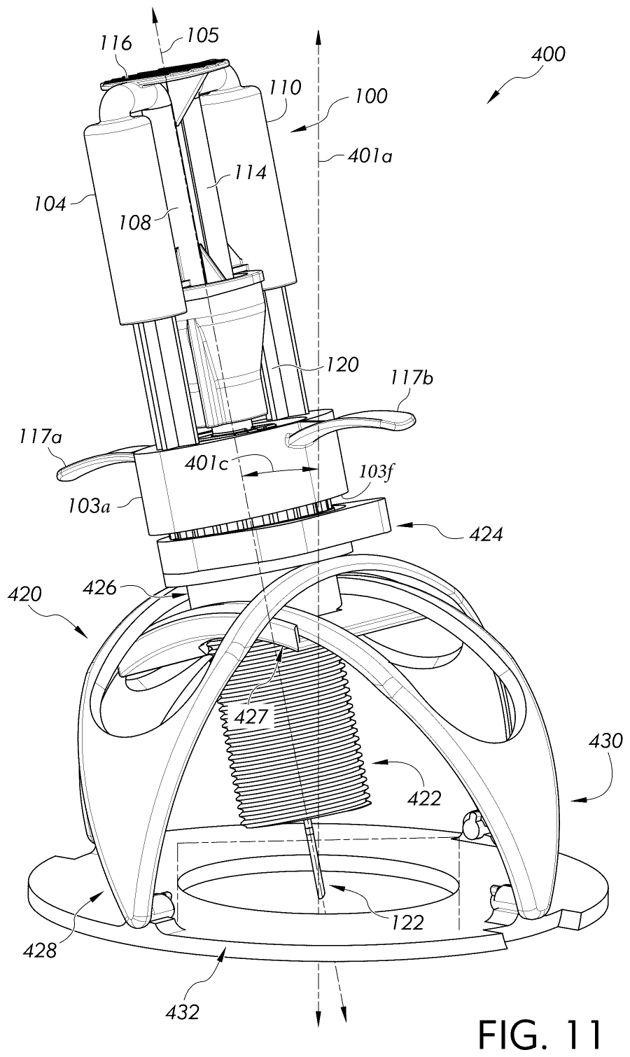

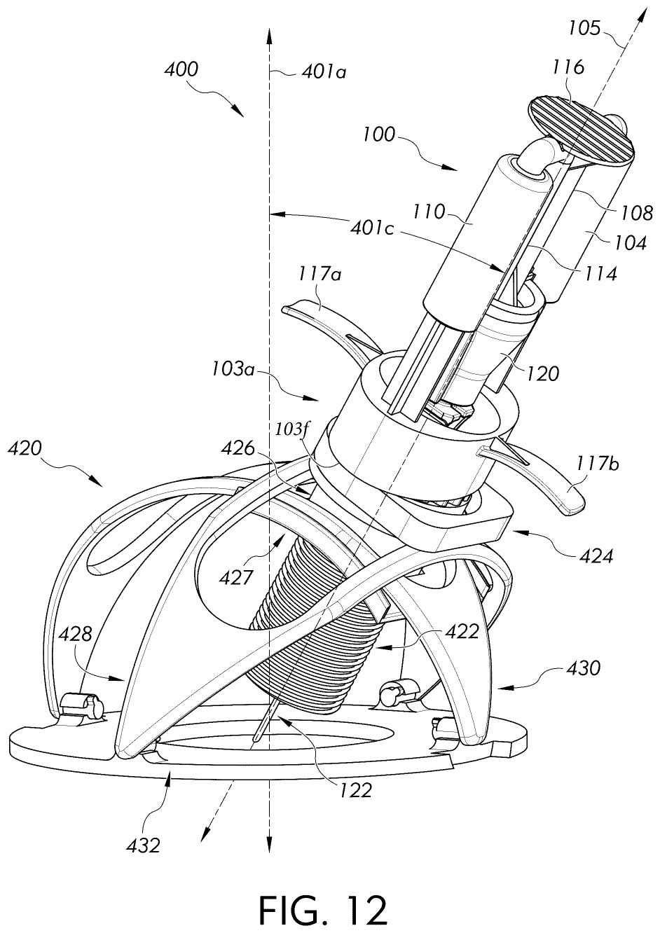

Referring now to FIGS. 11-12, a system 400 is shown having the component delivery system 100 and a guide assembly 420. Although depicted with component delivery system 100, any syringe assembly and/or needle assembly may be used with the guide assembly 420.

The component delivery system 100 is placed into the guide assembly 420. The guide assembly 420 is centered upon a guide axis 401a. When the medical professional is ready to dispense the multi-component sealant to the subject during a medical procedure and/or medical study, the guide assembly 420 is placed upon the skin of the subject so that the needle assembly 122 may enter the skin and translate towards the targeted treatment area.

The guide assembly 420 provides the medical professional control over the placement of the component delivery system 100 prior to injecting the multi-component sealant. The medical professional may control the depth of the component delivery system 100 using the guide assembly 420, so that they may determine a desired depth adjustment of the needle assembly 122 before entering the subject. The component delivery system 100 defines a center axis 105. The guide assembly 420 allows for depth adjustment along the center axis 105 of the component delivery system 100.

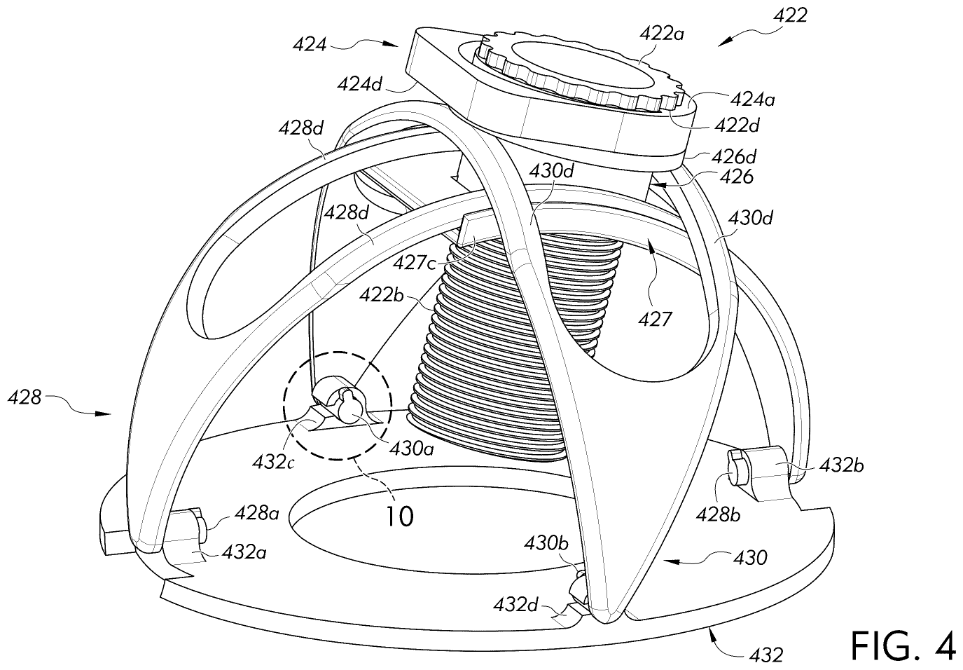

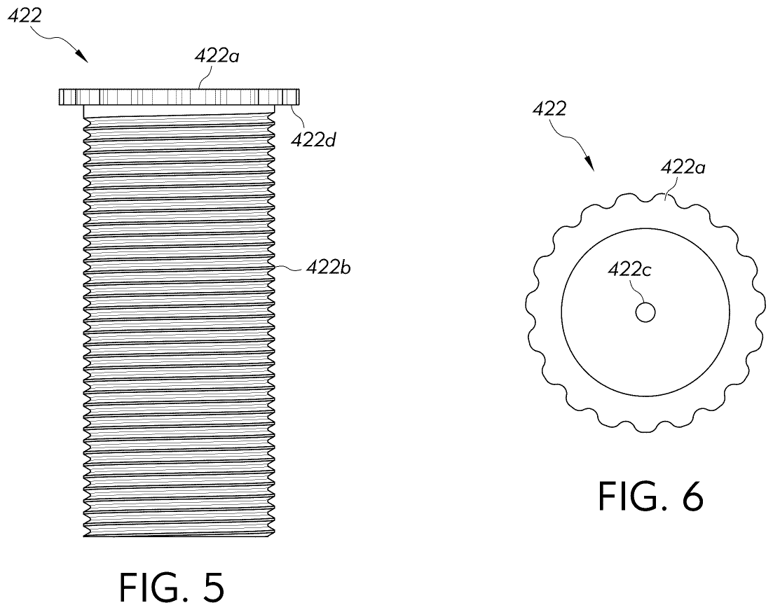

The medical professional may also determine an angle of the component delivery system 100 using the guide assembly 420, so that they may control an entry angle 401c of the needle assembly 122 before entering the subject. The entry angle 401c may be defined by comparing a deviation of the center axis 105 from the guide axis 401a. As depicted in FIG. 4, the entry angle 401c of the component delivery system 100 is depicted at a first position of the system 400. As depicted in FIG. 5, the guide assembly 420 is at a second position after being adjusted to achieve a different entry angle 401c.

Continuing to refer to FIGS. 11-12, the base 103a defines a base surface 103f. As discussed in greater detail herein, the guide assembly 420 includes depth adjustment component, an angular adjustment component, and a base 432. The depth adjustment component includes a threaded shaft 422. The threaded shaft 422 includes a first shaft surface 422a, a threaded portion 422b, a shaft outlet 422c, and a second shaft surface 422d (shown in FIGS. 5-6). The component delivery system 100 is removably coupled to the guide assembly 420 by placing the base 103a in contact with the first shaft surface 422a of the threaded shaft 422. The base surface 103f may define a flat profile, but other shapes are contemplated and possible. For example, the base surface 103f may have a protruding profile and first shaft surface 422a may be shaped and sized to define a receiving cavity to receive the protruding profile.

In embodiments, the base surface 103f includes engaging features (e.g., snap-fit connectors, fasteners) to removably secure the component delivery system 100 to the guide assembly 420. In this way, the component delivery system 100 is better located in the guide assembly 420 to minimize unintentional movement (e.g., slack) in the system 400. This is advantageous as it provides better control for the medical professional using the system 400 prior to injecting the multi-component sealant in the subject during the procedure.

When the component delivery system 100 is placed into the guide assembly 420, the needle assembly 122 of the component delivery system 100 extends along a portion of the threaded shaft 422 and continues to extend out of the shaft outlet 422c.

Referring now to FIG. 4, the depth adjustment component of the guide assembly 420 includes the threaded shaft 422, a collar 424, and a shaft platform 426. The angular adjustment component of the guide assembly 420 includes an arched platform 427, a first guide member 428, and a second guide member 430.

The threaded shaft 422 provides for the depth adjustment for the guide assembly 420. As discussed above, the threaded shaft 422 includes the first shaft surface 422a, the threaded portion 422b, the shaft outlet 422c, and the second shaft surface 422d. The second shaft surface 422d is positioned opposite to the first shaft surface 422a on a shaft head of the threaded shaft 422. As depicted the threaded portion 422b defines externally placed threads. In embodiments, the threaded portion 422b defines internally placed threads. The threaded shaft 422 may be constructed of metal, plastic, or of the like. The shaft outlet 422c is sized such that a variety of needle assembly gauges may be used.

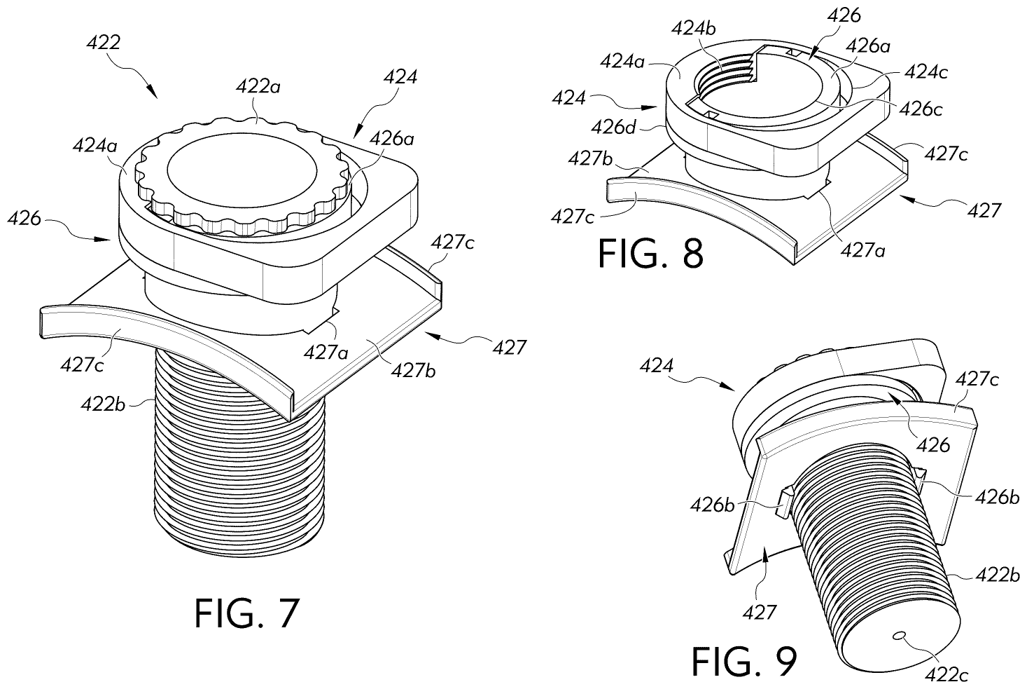

Referring now to FIGS. 7-9, the collar 424 includes a first collar surface 424a, internal threads 424b, a sleeve 424c, and a second collar surface 424d. The first collar surface 424a is opposed on an opposite side of the second collar surface 424d. When the threaded shaft 422 is assembled into the collar 424, the second shaft surface 422d is in contact with the first collar surface 424a. At a desired depth position, the threaded portion 422b of the threaded shaft 422 may engage the internal threads 424b of the collar 424. The internal threads 424b of the collar 424 may then lock the threaded shaft 422 in place. Additionally, the internal threads 424b allow for fine adjustments of the threaded shaft 422 by fastening the threaded shaft 422 along the center axis 105.

In this way, the medical professional may be able to adjust a depth placement of the component delivery system 100 by first sliding the threaded shaft 422 in the collar 424 in an adjustment position and then engaging the internal threads 424b of the collar 424 to the threaded portion 422b of the threaded shaft 422 in a locked position. Additionally, the medical professional may make fine-tune adjustments by fastening the threaded shaft 422 along the center axis 105. Regardless of the adjustments made to achieve the desired entry angle 401c, the depth of the needle assembly 122 to the skin of the subject is maintained. In embodiments where the threaded portion 422b of the threaded shaft 422 are placed internally, the threads of the collar 424 may be positioned on an external surface of the collar 424.

The shaft platform 426 includes a shaft surface 426a, engaging tabs 426b, a slot 426c, and a shaft outer surface 426d. The shaft platform 426 is partially positioned within the sleeve 424c of the collar 424 and is coupled to the collar 424. In embodiments, the collar 424 is integrated with the shaft platform 426. When the threaded shaft 422 is placed into the collar 424, it is simultaneously placed into the slot 426c of the shaft platform 426. The slot 426c extends through a depth of the shaft platform 426. An inner diameter of the slot 426c is shaped and sized such that it exceeds an outer diameter of the threaded portion 422b of the threaded shaft 422. In this way, the threaded shaft 422 may be slidably movable along a length of the shaft platform 426 and the collar 424 before engaging the internal threads 424b of the collar 424. This is advantageous as it allows for large depth adjustments to be made without needing to completely fasten or unfasten the threaded shaft 422 along the center axis 105. As depicted, the shaft outer surface 426d of the shaft platform 426 is circular, but other geometric profiles are contemplated and possible.

The arched platform 427 includes engagement slots 427a, an arched surface 427b, and arched railings 427c. The arched platform 427 encompasses an outer perimeter of the threaded portion 422b of the threaded shaft 422. The engagement slots 427a are formed within the arched surface 427b. The engaging tabs 426b of shaft platform 426 couple the shaft platform 426 to the arched platform 427 via the engagement slots 427a. The arched railings 427c are positioned on opposing sides of the arched surface 427b and extend at least partially along the opposing sides of the arched surface 427b.

In response to a force being applied upon the arched railings 427c, the arched platform 427 move in response in the direction of the applied force. As the arched platform 427 is coupled to the shaft platform 426, this force results in the translation of the shaft platform 426 and collar 424. This results in the translation of the component delivery system 100 that is positioned in the shaft platform 426 and the collar 424.

Referring back to FIG. 4, the first guide member 428 and the second guide member 430 are shown. The first guide member 428 and the second guide member 430 allow for the adjustment of the entry angle 401c of the component delivery system 100. The first guide member 428 and the second guide member 430 co-operate to achieve the entry angle 401c by each being able to rotate in a rotational axis.

A first end 428a of the first guide member 428 is coupled to a first side 432a of the base 432. A second end 428b of the first guide member 428 is coupled to a second side 432b of the base 432. The first side 432a of the base 432 and the second side 432b of the base 432 are positioned on opposing points of the base 432.

The first guide member 428 defines an arched profile between the first end 428a and the second end 428b. A first intermediate portion 428d of the first guide member 428 is positioned between the first end 428a and the second end 428b. The first intermediate portion 428d contacts the arched surface 427b of the arched platform 427. Additionally, the first intermediate portion 428d contacts the arched railings 427c of the arched platform 427. The first guide member 428 rotates in a first rotational axis that is perpendicular to the length of the first guide member 428 in response to receiving a force.

To adjust the entry angle 401c of the component delivery system 100, the medical professional may apply a force onto any of the components of the component delivery system 100 or the guide assembly 420. As the components of the guide assembly 420 are coupled to each other, the input force is ultimately transfer to the arched railings 427c of the arched platform 427. The force upon the arched railings 427c is transferred onto the first intermediate portion 428d of the first guide member 428. In response to receiving this force onto the first intermediate portion 428d, the first guide member 428 may rotate in the first rotational axis. The first guide member 428 may be constructed of plastic, metal, or any other suitable material.

A first end 430a of the second guide member 430 is coupled to a third side 432c of the base 432. A second end 430b of the second guide member 430 is coupled to a fourth side 432d of the base 432. The third side 432c of the base 432 and the fourth side 432d of the base 432 are positioned on opposing points of the base 432.

The second guide member 430 defines an arched profile between the first end 430a and the second end 430b. A second intermediate portion 430d of the second guide member 430 is positioned between the first end 430a and the second end 430b. The second intermediate portion 430d of the second guide member 430 contacts the second collar surface 424d of the collar 424. Additionally, the first intermediate portion 428d contacts the shaft outer surface 426d of the shaft platform 426. The second guide member 430 rotates in a second rotational axis that is perpendicular to the length of the second guide member 430 in response to receiving a force.

To adjust the entry angle 401c of the component delivery system 100, the medical professional may apply a force onto any of the components of the component delivery system 100 or the guide assembly 420. As the components of the guide assembly 420 are coupled to each other, the input force is ultimately transfer to the shaft outer surface 426d of the shaft platform 426. The force upon the shaft outer surface 426d is transferred onto the second intermediate portion 430d of the second guide member 430. In response to receiving this force onto the second intermediate portion 430d, the second guide member 430 may rotate in the second rotation axis. The second guide member 430 may be constructed of plastic, metal, or any other suitable material.

The first guide member 428 and the second guide member 430 co-operate to adjust the entry angle 401c of the component delivery system 100. The first guide member 428 is adjusted in the first rotation axis and the second guide member 430 is adjusted in the second rotational axis. Accordingly, the medical professional may move the first guide member 428 and/or the second guide member 430 to achieve the desired entry angle 401c of the component delivery system 100. The adjustment of either the first guide member 428 or the second guide member 430 does not change the depth of the threaded shaft 422. In this way, the medical professional has improved control of injecting the needle assembly 122 of the component delivery system 100 having both depth and angle control.

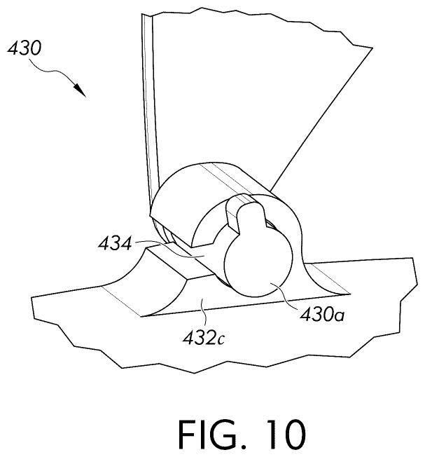

Referring now to FIG. 10, a detailed portion of the base 432 and the second guide member 430 are shown. Specifically, the third side 432c of the base 432 and the first end 430a of the second guide member 430 are shown. As illustrated, a shaft 434 of the first end 430a of the second guide member 430 extends within an aperture of the third side 432c and is coupled to the aperture of the third side 432c. The first end 430a is coupled to the third side 432c through an interference fit. In other words, the third side 432c secures the shaft 434 through friction. The third side 432c and the shaft 434 are shaped and sized such that the position of the first guide member 428 is static when the component delivery system 100 is being used or when the depth of the threaded shaft 422 is being adjusted.

The third side 432c and the shaft 434 are further shaped and sized such that the first guide member 428 is adjusted in response to receiving force having a rotational direction. The second side 432b of the second guide member 430 and the first and second ends 428a, 428b of the first guide member 428 are shaped and sized similarly to the first side 432a of the second guide member 430 fashion. Accordingly, each of the first end 428a and the second end 428b of the first guide member 428 and the first end 430a and second end 430b of the second guide member 430 may include a shaft 434 positioned within the respective base end.

Accordingly, the first side 432a, the second side 432b, and the fourth side 432d of the base 432 are shaped and sized similarly to the third side 432c.

The base 432 is centered upon the guide axis 401a and is placed upon the subject. The base 432 may be constructed of any suitable material for medical applications, such as plastic, metal, etc. As depicted, the base 432 defines a disc geometry. However, other geometries are considered and possible. For example, the base 432 may define a curved profile so that it may be better applied onto a curved surface of the subject (e.g., the subject's arm, the subject's leg). The base 432 defines a central aperture such that the needle assembly 122 of the component delivery system 100 may make contact with the skin of the subject.

Disclosed herein, is a component delivery system having a first body and a second body that includes each component of a multi-component sealant. When an engagement feature of the component delivery system is applied, the first body is moved towards the second body. This results in each component being dispensed from the first body to a mixing chamber. In the mixing chamber, the multi-component sealant begins to mix and is then dispensed from a needle assembly of the component delivery system.

Also disclosed herein is a guide assembly that may be used with the component delivery system or any other syringe/needle assembly. The guide assembly may control a depth and angle of a component delivery system prior to being injected into a subject. The depth control of the guide assembly moves the component delivery system along a single direction, while the angle control controls an entry angle of the component delivery system. The guide assembly is able to adjust the angle of the component delivery system while not effecting the depth of the component delivery system.

Summarizing, this disclosure may be considered to relate to the following aspects. In a first aspect, a medical sealant applicator device for delivery of a multi-component sealant including a first body having a base, a first syringe plunger, a second syringe plunger, and a longitudinal channel, laterally interposed between the first syringe plunger and the second syringe plunger. Each of the first syringe plunger and the second syringe plunger is oriented to extend in a first direction from the base. The first syringe plunger has a first free end having a first proximal plunger piston. The second syringe plunger having a second free end having a second proximal plunger piston. A second body of the medical sealant application device has a first syringe chamber, a second syringe chamber, and a longitudinal fluid chamber laterally interposed between the first syringe chamber and the second syringe chamber. The longitudinal fluid chamber has a proximal end and a distal end. The proximal end of the longitudinal fluid chamber being fluid communication with each of the first syringe chamber and the second syringe chamber. The distal end of the longitudinal fluid chamber has a distal needle connector port. The first syringe chamber of the second body has a first distal opening to slidably receive the first proximal plunger piston of the first syringe plunger of the first body. The second syringe chamber of the second body has a second distal opening to slidably receive the second proximal plunger piston of the second syringe plunger of the first body, and the distal needle connector port of the longitudinal fluid chamber of the second body is able to translate in the longitudinal channel of the first body in response to the second body longitudinally translating relative to the first body.

In a second aspect, in view of the first aspect, when a force is applied to the proximal end of a first syringe barrel and a second syringe barrel in a distal direction, the force simultaneously overcomes a biasing force of a first biasing assembly of the first body and a second biasing assembly of the medical sealant applicator device, thereby causing the first syringe plunger and the second syringe plunger to move in a proximal direction relative to the first syringe barrel, the second syringe barrel, and an elongate needle to move distally in the distal direction opposite the proximal direction away from the base.

In a third aspect or second aspect, in view of the first aspect, the base of the first body includes a distal aperture, wherein the longitudinal channel extends in the first direction from the distal aperture, and wherein the medical sealant applicator device further comprises a needle assembly having a connector hub and an elongate needle that extends from the connector hub, the connector hub being removably coupled to the distal needle connector port of the second body, wherein the elongate needle extends from the second body through the distal aperture of the base of the first body.

In a fourth aspect, accordingly to any of the earlier aspects, the second body comprises: a first u-shaped connector connecting the proximal end of the longitudinal fluid chamber in fluid communication with the first syringe chamber; and a second u-shaped connector connecting the proximal end of the longitudinal fluid chamber in fluid communication with the second syringe chamber.

In a fifth aspect, accordingly to any of the first to fourth aspects, the medical sealant applicator device comprises: includes a pair of finger-hold projections that laterally extend from the base of the first body.

In a sixth aspect, accordingly to any of the first to fifth aspects, the medical sealant applicator device comprises: a guide assembly to set a depth for advancement of the elongate needle.

In a seventh aspect, a medical sealant applicator device for delivery of a multi-component sealant, comprises: a first body, a second body, and a guide assembly. The first body has a base, a first syringe plunger, a second syringe plunger, and a longitudinal channel laterally interposed between the first syringe plunger and the second syringe plunger. Each of the first syringe plunger and the second syringe plunger is oriented to extend in a first direction from the base. The first syringe plunger has a first free end having a first proximal plunger piston. The second syringe plunger has a second free end having a second proximal plunger piston. The second body has a first syringe chamber, a second syringe chamber, and a longitudinal fluid chamber laterally interposed between the first syringe chamber and the second syringe chamber. The longitudinal fluid chamber has a proximal end and a distal end. The proximal end of the longitudinal fluid chamber being connected in fluid communication with each of the first syringe chamber and the second syringe chamber. The distal end of the longitudinal fluid chamber having a distal needle connector port. The first syringe chamber of the second body has a first distal opening to slidably receive the first proximal plunger piston of the first syringe plunger of the first body. The second syringe chamber of the second body has a second distal opening to slidably receive the second proximal plunger piston of the second syringe plunger of the first body. The distal needle connector port of the longitudinal fluid chamber of the second body is able to translate in the longitudinal channel of the first body in response to the second body longitudinally translating relative to the first body. The guide assembly sets a trajectory towards a targeted location and maintain the trajectory while advancing the needle assembly and injecting a component.

In an eighth aspect, according to the seventh aspect, when a force is applied to the proximal end of a first syringe barrel and a second syringe barrel in a distal direction, the force simultaneously overcomes a biasing force of a first biasing assembly of the first body and a second biasing assembly of the second body, thereby causing the first syringe plunger and the second syringe plunger to move in a proximal direction relative to the first syringe barrel and the second syringe plunger and an elongate needle to move distally in the distal direction opposite the proximal direction away from the base.

In a ninth aspect, according to the seventh aspect or the eighth aspect, the base of the first body includes a distal aperture, wherein the longitudinal channel extends in the first direction from the distal aperture, and wherein the medical sealant applicator device further comprises a needle assembly having a connector hub and an elongate needle that extends from the connector hub, the connector hub removably coupled to the distal needle connector port of the second body, wherein the elongate needle extends from the second body through the distal aperture of the base of the first body.

In a tenth aspect, according to any of the seventh to ninth aspects, the second body includes a first u-shaped connector connecting the proximal end of the longitudinal fluid chamber in fluid communication with the first syringe chamber and a second u-shaped connector connecting the proximal end of the longitudinal fluid chamber in fluid communication with the second syringe chamber.

In an eleventh aspect, according to any of the seventh aspect to the tenth aspect, a pair of finger-hold projections that laterally extend from the base of the first body.

In a twelfth aspect, according to any of the seventh aspect to the eleventh aspect, the guide assembly sets a depth for advancement of the elongate needle.

In a thirteenth aspect, a method includes setting a trajectory for a needle assembly through a pleural space towards a target at a known distance and maintain that trajectory while advancing the needle assemble. The method further includes advancing the needle assembly at a first rate through the pleural space to the known distance. The method further includes applying a force to a proximal end of the needle assemble in a distal direction, wherein the force simultaneously injects a substance at a second rate.

In a fourteenth aspect, according to the thirteenth aspect, the advancing of the needle assembly is simultaneous to the applying of the force.

In a fifteenth aspect, according to the thirteenth aspect or the fourteenth aspect, the first rate of the needle assembly is correlated to the second rate of injecting the substance.

In a sixteenth aspect, according to the fifteenth aspect, a correlation of the first rate of the needle assembly to the second rate of injecting the substance is modifiable.

In a seventeenth aspect, according to any of thirteenth aspect through the sixteenth aspect, a guide assembly advances the needle assembly further into a lung of a subject.

In an eighteenth aspect, a component delivery system, includes a needle assembly, an applicator, a first body, and a second body. The needle assembly has an elongate needle comprising a proximal end and a distal end spaced a distance apart from one another and defining at least one fluid path therebetween. The distal end comprising one or more fluid outlets coupled to the at least one fluid path. The applicator being fluidly coupled to the proximal end of the needle assembly. The applicator includes a first body comprising a first biasing assembly that is coupled to the needle assembly. The first body biasing the needle assembly in a proximal direction with respect to the applicator. The second body includes a syringe chamber having a needle connector port on a proximal end thereof that is fluidly coupled to the needle assembly, and a syringe plunger disposed within the syringe chamber at a distal end thereof and movable within the syringe chamber. The second biasing assembly that is coupled between the first body and the second body, the second biasing assembly biasing the syringe plunger at the distal end of the syringe chamber.

In a nineteenth aspect, according to the eighteenth aspect, when a force is applied to the proximal end of the syringe chamber in a distal direction, the force simultaneously overcomes a biasing force of the first biasing assembly and the second biasing assembly, thereby causing the syringe plunger to move in a proximal direction relative to the syringe chamber and the needle assembly to move distally in the distal direction opposite the proximal direction away from the first body.

In a twentieth aspect, according to the eighteenth aspect or the nineteenth aspect, the needle assembly further includes a connector hub removably coupled to the needle connector port, wherein the elongate needle extends from the second body through the first body.

In a twenty-first aspect, according to any one of the eighteenth aspect to the twentieth aspect, includes a pair of finger-hold projections that laterally extend from the first body.

In a twenty-second aspect, according to any one of the eighteenth aspect to the twenty-first aspect, includes a guide assembly to set a depth for advancement of the elongate needle.

In a twenty-third aspect, according to any one of the eighteenth aspect to the twenty-second aspect, further includes a guide assembly includes a depth adjustment component including a threaded shaft comprising a shaft head having a first shaft surface and a second shaft surface opposite the first shaft surface, the first shaft surface in contact with the component delivery system, a collar having an internally threaded portion threadingly engaged to the threaded shaft in a locked setting and a slot allowing for translation of the threaded shaft along a center axis of the guide assembly in an adjustment setting, a shaft platform in contact with the second shaft surface of the shaft head; and an arched platform coupled to the shaft platform; a base; and an angular adjustment component having a first guide member having a first end coupled to the base, a second end coupled to the base, and a first intermediate portion between the first end and the second end, the first intermediate portion disposed a distance from the base and contacting the arched platform; and a second guide member having a first end coupled to the base, a second end coupled to the base, and a second intermediate portion between the first end of the second guide member and the second end of the second guide member, the second intermediate portion disposed a distance from the base and contacting the shaft platform.

In a twenty-fourth aspect, according to the twenty-third aspect, the threaded shaft is movable along the center axis while in the adjustment setting.

In a twenty-fifth aspect, according to the twenty-third aspect, the depth adjustment component is movably coupled to the first guide member along a first rotational axis and wherein the second guide member is movably coupled along a second rotational axis perpendicular to the first rotational axis.

In a twenty-sixth aspect, according to any of the twenty-third aspect to the twenty-fifth aspect, the threaded shaft further includes a threaded portion defining an outer diameter, and the slot defines an inner diameter larger in size than the outer diameter.

In a twenty-seventh aspect, according to any of the twenty-third aspect to the twenty-sixth aspect, the elongate needle is disposed in a shaft outlet of the threaded shaft.

In a twenty-eight aspect, according to any of the twenty-third aspect to the twenty-sixth aspect, the first end and the second end are rotatably coupled to the base, thereby facilitating rotation of the first guide member relative to the base.

In a twenty-ninth aspect, according to any of the twenty-third aspect twenty-eighth aspect, the first end of the second guide member and the second end of the second guide member are rotatably coupled to the base, thereby facilitating rotation of the second guide member relative to the base.

In a thirtieth aspect, according to any of the twenty-third aspect to the twenty-ninth aspect, the first intermediate portion and the second intermediate portion are disposed between a surface of the shaft platform and a surface of the arched platform.

In a thirty-first aspect, according to any of the twenty-third aspect to the thirtieth aspect, the base further includes a first side and a second side diametrically opposed to the first side; a third side and a fourth side diametrically opposed to the third side; the first side coupled to the first end of the first guide member; the second side coupled to the second end of the first guide member; the third side coupled to the first end of the second guide member; and the fourth side coupled to the second end of the second guide member.

In a thirty-second aspect, a device includes a needle assembly for which a trajectory is set through a pleural space towards a target at a known distance and maintaining the trajectory while advancing the needle assembly. The needle assembly is advanced at a first rate through the pleural space to the known distance. A force is applied onto a proximal end of the needle assembly in a distal direction, wherein the force simultaneously injects a substance at a second rate.

For the purposes of describing and defining the present disclosure it is noted that the term “substantially” is used herein to represent the inherent degree of uncertainty that may be attributed to any quantitative comparison, value, measurement, or other representation. The term “substantially” is used herein also to represent the degree by which a quantitative representation may vary from a stated reference without resulting in a change in the basic function of the subject matter at issue. As such, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something slightly less than exact.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A medical sealant applicator device for delivery of a multi-component sealant, comprising:
a first body having a base, a first syringe plunger, a second syringe plunger, and a longitudinal channel, laterally interposed between the first syringe plunger and the second syringe plunger, wherein each of the first syringe plunger and the second syringe plunger is oriented to extend in a first direction from the base, the first syringe plunger having a first free end having a first proximal plunger piston, the second syringe plunger having a second free end having a second proximal plunger piston;
a second body having a first syringe chamber, a second syringe chamber, and a longitudinal fluid chamber laterally interposed between the first syringe chamber and the second syringe chamber, the longitudinal fluid chamber having a proximal end and a distal end, the proximal end of the longitudinal fluid chamber being connected in fluid communication with each of the first syringe chamber and the second syringe chamber, the distal end of the longitudinal fluid chamber having a distal needle connector port, wherein:
the first syringe chamber of the second body has a first distal opening to slidably receive the first proximal plunger piston of the first syringe plunger of the first body,
the second syringe chamber of the second body has a second distal opening to slidably receive the second proximal plunger piston of the second syringe plunger of the first body, and
the distal needle connector port of the longitudinal fluid chamber of the second body is able to translate in the longitudinal channel of the first body in response to the second body longitudinally translating relative to the first body.

2. The medical sealant applicator device of claim 1, wherein, when a force is applied to the proximal end of a first syringe barrel and a second syringe barrel in a distal direction, the force simultaneously overcomes a biasing force of a first biasing assembly of the first body and a second biasing assembly of the medical sealant applicator device, thereby causing the first syringe plunger and the second syringe plunger to move in a proximal direction relative to the first syringe barrel, the second syringe barrel, and an elongate needle to move distally in the distal direction opposite the proximal direction away from the base.

3. The medical sealant applicator device according to claim 1, wherein the base of the first body includes a distal aperture, wherein the longitudinal channel extends in the first direction from the distal aperture, and wherein the medical sealant applicator device further comprises a needle assembly having a connector hub and an elongate needle that extends from the connector hub, the connector hub being removably coupled to the distal needle connector port of the second body, wherein the elongate needle extends from the second body through the distal aperture of the base of the first body.

4. The medical sealant applicator device according to claim 1, wherein the second body comprises:
a first u-shaped connector connecting the proximal end of the longitudinal fluid chamber in fluid communication with the first syringe chamber; and
a second u-shaped connector connecting the proximal end of the longitudinal fluid chamber in fluid communication with the second syringe chamber.

5. The medical sealant applicator device according to claim 1, comprising a pair of finger-hold projections that laterally extend from the base of the first body.

6. The medical sealant applicator device according to claim 1, comprising a guide assembly to set a depth for advancement of the elongate needle.

7. The medical sealant applicator device according to claim 3, comprising:
a guide assembly to set a trajectory towards a targeted location and maintains the trajectory while advancing the needle assembly and injecting a component.

8. (canceled)

9. (canceled)

10. (canceled)

11. (canceled)

12. (canceled)

13. A method comprising:
setting a trajectory for a needle assembly through a pleural space towards a target at a known distance and maintaining the trajectory while advancing the needle assembly;
advancing the needle assembly at a first rate through the pleural space to the known distance; and
applying a force to a proximal end of the needle assembly in a distal direction, wherein the force simultaneously injects a substance at a second rate.

14. The method according to claim 13, wherein the advancing of the needle assembly is simultaneous to the applying of the force.

15. The method according to claim 13, wherein the first rate of the needle assembly is correlated to the second rate of injecting the substance.

16. The method according to claim 15, wherein a correlation of the first rate of the needle assembly to the second rate of injecting the substance is modifiable.

17. The method according to claim 13, wherein a guide assembly advances the needle assembly further into a lung of a subject.

18. A component delivery system, comprising:
a needle assembly having an elongate needle comprising a proximal end and a distal end spaced a distance apart from one another and defining at least one fluid path therebetween, the distal end comprising one or more fluid outlets coupled to the at least one fluid path; and
an applicator fluidly coupled to the proximal end of the needle assembly, the applicator comprising:
a first body comprising a first biasing assembly that is coupled to the needle assembly, the first body biasing the needle assembly in a proximal direction with respect to the applicator,
a second body comprising:
a syringe chamber having a needle connector port on a proximal end thereof that is fluidly coupled to the needle assembly, and
a syringe plunger disposed within the syringe chamber at a distal end thereof and movable within the syringe chamber, and
a second biasing assembly that is coupled between the first body and the second body, the second biasing assembly biasing the syringe plunger at the distal end of the syringe chamber.

19. The component delivery system according to claim 18, wherein, when a force is applied to the proximal end of the syringe chamber in a distal direction, the force simultaneously overcomes a biasing force of the first biasing assembly and the second biasing assembly, thereby causing the syringe plunger to move in a proximal direction relative to the syringe chamber and the needle assembly to move distally in the distal direction opposite the proximal direction away from the first body.

20. The component delivery system according to claim 18, wherein the needle assembly further comprises a connector hub removably coupled to the needle connector port, wherein the elongate needle extends from the second body through the first body.

21. The component delivery system according to claim 18, comprising a pair of finger-hold projections that laterally extend from the first body.

22. (canceled)

23. The component delivery system according to claim 18, further comprising a guide assembly to set a depth for advancement of the elongate needle comprising:
a depth adjustment component comprising:
a threaded shaft comprising a shaft head having a first shaft surface and a second shaft surface opposite the first shaft surface, the first shaft surface in contact with the component delivery system;
a collar comprising an internally threaded portion threadingly engaged to the threaded shaft in a locked setting and a slot allowing for translation of the threaded shaft along a center axis of the guide assembly in an adjustment setting;
a shaft platform in contact with the second shaft surface of the shaft head; and
an arched platform coupled to the shaft platform;
a base; and
an angular adjustment component comprising:
a first guide member having a first end coupled to the base, a second end coupled to the base, and a first intermediate portion between the first end and the second end, the first intermediate portion disposed a distance from the base and contacting the arched platform; and
a second guide member having a first end coupled to the base, a second end coupled to the base, and a second intermediate portion between the first end of the second guide member and the second end of the second guide member, the second intermediate portion disposed a distance from the base and contacting the shaft platform.

24. The component delivery system according to claim 23, wherein the threaded shaft is movable along the center axis while in the adjustment setting.

25. The component delivery system according to claim 23, wherein the depth adjustment component is movably coupled to the first guide member along a first rotational axis and wherein the second guide member is movably coupled along a second rotational axis perpendicular to the first rotational axis.

26. The component delivery system according to claim 23, wherein:
the threaded shaft further comprises a threaded portion defining an outer diameter, and
the slot defines an inner diameter larger in size than the outer diameter.

27. The component delivery system according to claim 23, wherein the elongate needle is disposed in a shaft outlet of the threaded shaft.

28. (canceled)

29. The component delivery system according to claim 23, wherein the first end of the second guide member and the second end of the second guide member are rotatably coupled to the base, thereby facilitating rotation of the second guide member relative to the base.

30. The component delivery system according to claim 23, wherein the first intermediate portion and the second intermediate portion are disposed between a surface of the shaft platform and a surface of the arched platform.

31. The component delivery system according to claim 23, wherein the base further comprises:
a first side and a second side diametrically opposed to the first side;
a third side and a fourth side diametrically opposed to the third side;
the first side coupled to the first end of the first guide member;
the second side coupled to the second end of the first guide member;
the third side coupled to the first end of the second guide member; and
the fourth side coupled to the second end of the second guide member.

32. (canceled)
